(19) United States
(12) Patent Application Publication
(10) Pub. No.: US 2026/0013940 A1
(43) Pub. Date: Jan. 15, 2026

(54) METHOD AND SYSTEM FOR VISUAL SUPPORT IN A MEDICAL INTERVENTION ON A HOLLOW ORGAN

(71) Applicant: Siemens Healthineers AG, Forchheim (DE)

(72) Inventors: Marcus Pfister, Bubenreuth (DE); Philipp Roser, Erlangen (DE)

(21) Appl. No.: 18/772,105

(22) Filed: Jul. 12, 2024

(30) Foreign Application Priority Data
Jul. 13, 2023 (DE) 10 2023 206 678.5

(51) Int. Cl. A61B 34/10; A61B 6/00; A61B 6/02; A61B 6/50; G06T 7/33; G06T 11/00; G16H 30/20; G16H 30/40

(52) U.S. Cl. CPC A61B 34/10; A61B 6/025; A61B 6/504; A61B 6/5229; G06T 7/337; G06T 11/006; G16H 30/20; G16H 30/40; A61B 2034/105; A61B 2034/107; G06T 2207/10016; G06T 2207/10081; G06T 2207/10116; G06T 2207/30101; G06T 2207/30172

(57) ABSTRACT

For visual support in a medical intervention on a hollow organ of a patient, reference data that includes a reference representation of the hollow organ is obtained, an X-ray projection image of the hollow organ is generated, and items of overlay information are determined based on the reference data. The reference data includes items of planning information relating to a location of a characteristic feature of the hollow organ in the reference representation. A reference volume is determined in the reference representation as a function of the items of planning information. A reconstruction of a volume of the patient corresponding to the reference volume is generated using a tomosynthesis method. Registration data is generated in that the reference volume is registered in relation to the reconstruction and the items of overlay information are overlaid on the X-ray projection image as a function of the registration data.

This application claims the benefit of German Patent Application No. DE 10 2023 206 678.5, filed on Jul. 13, 2023, which is hereby incorporated by reference in its entirety.

### BACKGROUND

The present embodiments relate to visual support in a medical intervention on a hollow organ of a patient.

An abdominal aortic aneurysm (AAA) is an enlargement of the abdominal aorta that, for example, is in a region between the branching of the aorta and the vascular outlets to the kidneys. AAA may result in a life-threatening rupture in which large volumes of blood may pass into the abdominal cavity.

In order to treat an AAA, for example, interventions may be carried out by a catheter. With the catheter, a vessel transplant is introduced via an incision in the groin, expanded, and positioned at the site of the aneurysm. The correct positioning of the vessel transplant may be monitored during the intervention (e.g., using fluoroscopy).

One problem in this connection is that the aorta may potentially be only very poorly identified in X-ray projection images if no contrast medium is used in the imaging. A pre-operative CT image dataset may therefore be registered with the system used for the fluoroscopy in order that the aorta and/or items of planning information from the CT image dataset are superimposed in the X-ray projection images in fluoroscopy in order to visually support medical staff during the implantation process.

Against this background, document DE 10 2011 080 588 A1 describes a method for 3D/3D registration of a first 3D image dataset that was recorded with a computer tomograph, and a second 3D image dataset that was recorded with a C-arm system. A body part is segmented or a model thereof is generated from the first 3D image dataset. First, a rough registration of the first 3D image dataset with the second 3D image dataset is carried out. Using this as a starting point, a fine registration is carried out based on the segmented body part or its model in which only the segmented body part or the model thereof is spatially aligned with the body part from the second 3D image dataset.

One drawback in this connection is the great effort that is necessary to generate the second 3D image dataset since, for example, a very large number of individual 2D-images are to be recorded from different directions for this purpose in order to reconstruct the second 3D image dataset therefrom using computed tomography techniques.

Although the problem has been represented based on visual support for implantation of a vessel transplant in the case of an AAA, analog situations may also result in the case of other medical interventions (e.g., interventions on hollow organs).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, medical staff may be visually supported in a medical intervention on a hollow organ of a patient with reduced effort.

The present embodiments are based on the idea of registering a reference volume of a pre-operative reference representation in relation to a corresponding reconstruction that is obtained by tomosynthesis. The reference volume is defined based on items of planning information, and the tomosynthesis is carried out accordingly.

According to one aspect of the present embodiments, a method for visual support (e.g., of visual support by way of fluoroscopy, such as of visual support of medical staff) is provided in the case of a medical intervention on a hollow organ of a patient. Reference data is obtained, for example, by at least one computing unit that includes a pre-operative reference representation (e.g., a three-dimensional reference representation) of the hollow organ. An X-ray projection image of the hollow organ is generated (e.g., during the intervention, such as by actuation of an X-ray imaging modality by the at least one computing unit). Items of overlay information are determined, for example, by the at least one computing unit, based on the reference data. An overlay image is displayed by a display device (e.g., actuated by the at least one computing unit). The overlay image corresponds to the X-ray projection image overlaid with the items of overlay information.

The reference data also includes items of planning information relating to at least one respective location of at least one characteristic feature of the hollow organ in the reference representation. A reference volume is determined in the reference representation as a function of the items of planning information (e.g., as a function of the respective location of the at least one characteristic feature). A reconstruction of a volume of the patient corresponding to the reference volume is generated (e.g., by actuation of the X-ray imaging modality by the at least one computing unit) using a tomosynthesis method (e.g., during the intervention; as a function of the reference volume in the reference representation). Registration data is generated, for example, by the at least one computing unit, in that the reference volume is registered in relation to the reconstruction. In order to generate the overlay image, the items of overlay information are overlaid on the X-ray projection image as a function of the registration data.

The reference data may be obtained by at least one computing unit, for example, in that the reference data is provided on a computer-readable memory medium.

The medical intervention may be, for example, a diagnostic intervention and/or a therapeutic intervention or the like. For example, the medical intervention may be an intervention for implantation of a vessel transplant, also referred to as a stent or stent graft, for example, in order to treat an abdominal aortic aneurysm (AAA). In the latter case, the hollow organ is therefore the abdominal aorta of the patient. The medical intervention or other surgical measures or the like may not be part of the method of the present embodiments, however.

The reference representation is, for example, a three-dimensional representation of at least the hollow organ or a corresponding pre-operative three-dimensional model of at least the hollow organ determined by an imaging method before the medical intervention (e.g., pre-operatively). For example, the reference representation may be a three-dimensional computed tomography (CT) reconstruction.

The X-ray projection image and the reconstruction of the volume corresponding to the reference volume and the reference representation are not necessarily generated by the same X-ray imaging modality even if the reference representation is likewise generated using X-ray imaging. For example, the reference representation may be generated by a CT system, and the X-ray imaging modality for generating the X-ray projection image and for carrying out the tomosynthesis method may be a C-arm X-ray system (e.g., a C-arm X-ray angiography system).

Here and below, a location may be understood in such a way that the location includes a corresponding position (e.g., three-dimensional position) and a corresponding orientation (e.g., three-dimensional orientation). The items of planning information include items of information relating to the location of the at least one characteristic feature. For example, the items of planning information for each characteristic feature of the at least one characteristic feature contains items of information relating to the respective location of the characteristic feature. The reference data may also include items of planning information relating to at least one respective size and/or form of the at least one characteristic feature in the reference representation. The reference volume may also be as determined as a function of the respective size and/or form of the at least one characteristic feature. The items of planning information may also include, for example, a segmentation of the hollow organ.

One characteristic feature of the hollow organ may be an anatomical feature (e.g., a structure on a vessel wall of the hollow organ or, if the hollow organ is a vessel, a vascular outlet, such as a point on the vessel wall at which the vessel meets a further vessel, or a vascular bifurcation, etc.). One characteristic feature of the hollow organ may also be a geometric feature (e.g., a geometric body) that encloses or partially encloses the hollow organ, or, if the hollow organ is a vessel, a central line (e.g., a line running centrally through the vessel). If the vessel is viewed as a tube with approximately circular cross-section, the central line then always runs, for example, through the corresponding center of the circle. A cross-sectional area of the vessel may also be a characteristic feature of the hollow organ.

For example, a sequence of further X-ray projection images is generated in the tomosynthesis method, where each X-ray projection image of the sequence at least partially represents the hollow organ according to another predefined projection direction respectively. A three-dimensional reconstruction of a volume region of the patient, referred to as the tomosynthesis volume, may then be generated based on the X-ray projection images of the sequence (e.g., by the inverse Radon transform).

The tomosynthesis method therefore has commonalities with a CT method, where in the case of a CT method, a recording angle range of at least 180° of the projection directions is always run through; in the case of a tomosynthesis method, a recording angle range of the projection directions that is less than 180° is run through. From this smaller recording angle range, it follows that the tomosynthesis volume has a thickness that is smaller than in the case of a CT method. The tomosynthesis volume corresponds to a volume limited by two mutually parallel planes that may also be referred to as the planar slice, with a slice thickness of the planar slice corresponding to the spacing of the two parallel planes from each other.

The greater the recording angle range run through by the projection directions is, the greater the slice thickness is. For example, the slice thickness may lie in the order of magnitude of 1 cm up to a few 10 cm (e.g., up to 20 cm).

While the slice thickness of the tomosynthesis volume is defined by the recording angle range, its location is given by the position of the isocenter of the X-ray imaging modality and a reference recording direction, which corresponds to a central reference angle of the recording angle range. The parallel planes that limit the tomosynthesis volume are, for example, perpendicular to the reference recording direction, and a center of the tomosynthesis volume is situated in the isocenter of the X-ray imaging modality.

In one embodiment, the reference volume is defined based on the reference representation using the items of planning information. For example, the reference volume is similarly defined as a planar slice, like the tomosynthesis volume. If the location and slice thickness of the reference volume are known, the tomosynthesis method may thus be carried out such that, for example, via appropriate selection or setting of the recording angle range, the reference recording direction, and the isocenter, the resulting tomosynthesis volume is the volume of the patient corresponding to the reference volume.

For example, no exact registration of the reference representation or of the reference volume in relation to the X-ray imaging modality or the patient in advance is necessary for this. It may, however, be advantageous to carry out a corresponding rough registration or pre-registration in order to reduce the computing effort for generating the registration data or to increase the reliability of the optimization methods optionally involved in this connection.

The reference volume is then registered using a known registration method (e.g., a 3D/3D registration method) in relation to the reconstruction in order to generate the registration data. The registration data therefore indicates, for example, how individual voxels of the reconstruction have to be transformed (e.g., rotated and/or shifted) in order to convert the individual voxel to a corresponding voxel of the reference volume, or vice versa. The registration data may thus be used to transform the items of overlay information that likewise originate from the reference data accordingly, so that the items of overlay information are consistent with the X-ray projection image. That the items of overlay information are overlaid on the X-ray projection image as a function of the registration data may therefore be understood, for example, such that the items of overlay information are transformed in accordance with the registration data and the transformed items of overlay information are overlaid on the X-ray projection image, or that the X-ray projection image is transformed in accordance with the registration data and the items of overlay information are overlaid on the transformed X-ray projection image.

The items of overlay information may include a two-dimensional representation of the hollow organ or other organs of the patient that is obtained from the three-dimensional reference representation. Alternatively or in addition, the items of overlay information may also include two-dimensional representations of the characteristic features or other items of planning information.

Medical staff may therefore be visually supported via the overlay image in the case of medical intervention on the hollow organ by the method of the present embodiments, with a very accurate registration of the items of overlay information in relation to the X-ray projection image being achieved without a complete CT reconstruction and/or administering of contrast medium being necessary for registration.

According to at least one embodiment, the reference volume is determined as a planar slice. A slice thickness of the planar slice and a location of the planar slice in the reference representation are determined for determining the reference volume (e.g., therefore, with respect to the reference representation).

As explained above, the volume corresponding to the reference volume may therefore be reconstructed by a tomosynthesis with a particular recording angle range. The specific defining of the location of the planar slice and the slice thickness depends, for example, on the hollow organ and the intervention to be carried out.

In some embodiments, where the hollow organ is a vessel (e.g., a blood vessel), the at least one characteristic feature contains the central line.

In some embodiments, the reference volume is determined such that a center of the reference volume lies on the central line. The center of the reference volume is, for example, a point that is equidistantly spaced apart from the two parallel planes of the planar slice.

This achieves that an optimally relevant region of the hollow organ lies in the reference volume and is encompassed by the reconstruction.

According to at least one embodiment, the at least one characteristic feature contains a first vascular outlet on the vessel. Further, the at least one characteristic feature may contain a second vascular outlet on the vessel.

According to at least one embodiment, the reference volume is determined such that the reference volume at least partially includes the first vascular outlet or the second vascular outlet (e.g., at least partially includes the first vascular outlet and the second vascular outlet, respectively).

It is consequently achieved that an optimally relevant region of the hollow organ lies in the reference volume and is encompassed by the reconstruction.

For example, when the hollow organ is the abdominal aorta and the intervention is an implantation of a vessel transplant in the abdominal aorta, the reference volume may be determined such that the reference volume respectively at least partially includes the first vascular outlet and the second vascular outlet and the center of the reference volume lies on the central line.

According to at least one embodiment, a parameter set for carrying out the tomosynthesis method is determined as a function of the reference volume (e.g., as a function of the location and slice thickness of the planar slice). The tomosynthesis method is then carried out in accordance with the parameter set.

According to at least one embodiment, the parameter set includes the recording angle range, and the recording angle range is determined as a function of the slice thickness (e.g., using a known correlation between recording angle range and tomosynthesis slice thickness).

It is thus possible to achieve that the slice thickness of the reconstructed volume matches the slice thickness of the reference volume.

According to at least one embodiment, the parameter set includes the reference recording direction corresponding to a predefined reference angle of the recording angle range, and the reference recording direction is determined as a function of the location (e.g., the orientation) of the planar slice of the reference volume (e.g., with respect to the reference representation).

It is thus possible to achieve that the orientation of the reconstructed volume with respect to the patient or the hollow organ matches the orientation of the reference volume with respect to the reference representation of the hollow organ.

According to at least one embodiment, the parameter set includes the isocenter position (e.g., the X-ray imaging modality or the tomosynthesis method), and the isocenter position is determined as a function of the location (e.g., the position) of the planar slice of the reference volume (e.g., with respect to the reference representation).

It is thus possible to achieve that the position of the reconstructed volume with respect to the patient or with respect to the hollow organ matches the position of the reference volume with respect to the reference representation of the hollow organ.

According to at least one embodiment, a sequence of further X-ray projection images is generated for carrying out the tomosynthesis method, where each X-ray projection image of the sequence at least partially represents the hollow organ according to another predefined projection direction, respectively. The projection directions of the X-ray projection images of the sequence are characterized by a respective angle within the recording angle range in a plane, where the reference recording direction lies in the plane. The reconstruction is generated based on the X-ray projection images of the sequence.

The reconstruction is generated based on the further X-ray projection images of the sequence using a known reconstruction algorithm for tomosynthesis (e.g., based on the inverse Radon transform). The total number of further X-ray projection images of the sequence may lie in the order of magnitude of 10 to a number of 10s (e.g., several 10s; in the range [10, 50]).

The tomosynthesis method is carried out, for example, without administering contrast medium. The sequence of further X-ray projection images is therefore generated while there is no contrast medium, or no significant quantity of contrast medium, in the hollow organ.

A 3D/3D registration of the reconstruction in relation to the reference volume may consequently be carried out, and a high level of accuracy in the overlaying of the items of overlay information may be achieved thereby; the effort for the registration may still be kept much lower than in alternative methods (e.g., an intra-operative CT reconstruction).

The sequence corresponds, for example, to a temporal or chronological sequence of the further X-ray projection images, with the total number of the further X-ray projection images of the sequence being predefined. That each further X-ray projection image of the sequence at least partially represents the at least one hollow organ in accordance with another predefined projection direction, respectively, may be comprehended, for example, such that the individual projection directions are predefined for all further X-ray projection images of the sequence and differ from one another, respectively, for all further X-ray projection images of the sequence. The projection directions of two different further X-ray projection images of the sequence are therefore always different from one another.

The reference angle may be, for example, equal to zero, so the recording angle range is, for example, [−α/2, α/2] with α<180°. The recording angle range may also be defined as [0, α] with α<180°, with the reference angle then being α/2. The angle α may also be comprehended as the maximum angle difference of the recording angle range.

According to at least one embodiment, the maximum angle difference according to the recording angle range is less than 180° (e.g., less than or equal to 90°, less than or equal to 60°, or less than or equal to) 45°.

This results, for example, in a much less effort than, for example, in the case of an intra-operative CT reconstruction.

According to at least one embodiment, a pre-registration of the reference representation in relation to the patient is carried out before carrying out the tomosynthesis method (e.g., during the intervention).

The reliability of registration of the reference volume in relation to the reconstruction may consequently be improved. Known algorithms may likewise be used for pre-registration.

According to at least one embodiment, a first reference X-ray projection image of the hollow organ is generated, and the reference representation is registered in relation to the first reference X-ray projection image for carrying out the pre-registration.

A pre-registration may thus be achieved particularly easily.

According to at least one embodiment, a second reference X-ray projection image of the hollow organ is generated for carrying out the pre-registration, where a projection direction for generating the first reference X-ray projection image and a projection direction for generating the second reference X-ray projection image are different from one another. The reference representation is registered in relation to the first further X-ray projection image and the second reference X-ray projection image.

The accuracy of the pre-registration, and therewith the reliability of the registration of the reference volume in relation to the reconstruction, may thus be improved.

According to a further aspect of the present embodiments, a system for visual support in a medical intervention on a hollow organ of a patient is disclosed. The system has an X-ray imaging modality that is configured to generate an X-ray projection image of the hollow organ (e.g., actuated by at least one computing unit of the system). The at least one computing unit is configured to obtain reference data that includes a reference representation of the hollow organ, to determine items of overlay information based on the reference data, and to generate an overlay image that corresponds to the X-ray projection image overlaid with the items of overlay information.

The reference data includes items of planning information relating to a location of at least one characteristic feature of the hollow organ in the reference representation. The at least one computing unit is configured to determine a reference volume in the reference representation as a function of the items of planning information. The at least one computing unit is configured to actuate the X-ray imaging modality for carrying out a tomosynthesis method and to generate a reconstruction of a volume of the patient corresponding to the reference volume based on a result or intermediate result of the tomosynthesis method. The at least one computing unit is configured to generate registration data, to register the reference volume in relation to the reconstruction for this, and to overlay the items of overlay information on the X-ray projection image as a function of the registration data.

The result or intermediate result of the tomosynthesis method may be given, for example, by the sequence of further X-ray projection images, as described above.

The X-ray imaging modality has, for example, an X-ray source, an X-ray detector, and a drive apparatus for positioning the X-ray source and the X-ray detector with respect to the hollow organ corresponding to different projection directions.

The at least one computing unit is configured, for example, to actuate the drive apparatus and the X-ray source for generating the X-ray projection image and for carrying out the tomosynthesis method. The at least one computing unit is configured to obtain corresponding detector signals from the X-ray detector, and to generate the X-ray projection image or the sequence of further X-ray projection images on the basis thereof.

A computing unit may, for example, be taken to be a data processing device that contains a processing circuit. The computing unit may therefore process, for example, data for carrying out computing operations. These optionally also include operations to carry out indexed instances of access to a data structure (e.g., a look-up table (LUT)).

The computing unit may contain, for example, one or more computers, one or more microcontrollers, and/or one or more integrated circuits (e.g., one or more application-specific integrated circuit(s) (ASICs), one or more field-programmable gate array(s) (FPGA), and/or one or more system(s) on a chip (SoC)). The computing unit may also include one or more processors (e.g., one or more microprocessors, one or more central processing units (CPU), one or more graphics processing units (GPU), and/or one or more signal processors, such as one or more digital signal processors (DSP)). The computing unit may also include a physical or a virtual network of computers or other of said units.

In different example embodiments, the computing unit includes one or more hardware and/or software interfaces and/or one or more memory units.

A memory unit may be configured as a volatile data memory (e.g., as a dynamic random access memory (DRAM) or static random access memory (SRAM), a non-volatile data memory, such as a read-only memory (ROM), a programmable read-only memory (PROM), an erasable programmable read-only memory (EPROM), an electrically erasable programmable read-only memory (EEPROM), a flash memory or flash EEPROM, a ferroelectric random access memory (FRAM), a magnetoresistive random access memory (MRAM), or as a phase-change random access memory (PCRAM)).

The X-ray source and the X-ray detector are, for example, always positioned and/or oriented in the same way relative to one another, independently of the respective projection direction. The X-ray source and the X-ray detector may, for example, be rigidly connected to one another (e.g., via a C-arm or the like). The drive apparatus includes, for example, one or more motors or gear mechanisms for moving the X-ray source and the X-ray detector (e.g., simultaneously) in order to set the individual projection directions.

The X-ray imaging modality may be embodied, for example, as a C-arm X-ray angiography system.

According to at least one embodiment, the system includes a display device, and the at least one computing unit is configured to actuate the display device for displaying the overlay image.

Further embodiments of the system follow directly from the different embodiments of the method, and vice versa. For example, individual features and corresponding explanations and advantages with respect to the different embodiments in relation to the method may be analogously transferred to corresponding embodiments of the system. For example, the system of the present embodiments is configured or programmed for carrying out a method of the present embodiments. For example, the system of the present embodiments carries out the method of the present embodiments.

According to a further aspect of the present embodiments, a computer program with commands is disclosed. On execution of the commands by a system of the present embodiments (e.g., by the at least one computing unit of the system), the commands prompt the system to carry out a method of the present embodiments.

The commands may be present, for example, as program code. The program code may be provided, for example, as binary code or Assembler and/or as a source code of a programming language (e.g., C) and/or as program script (e.g., Python).

According to a further aspect of the present embodiments, a computer-readable storage medium is disclosed that stores a computer program of the present embodiments.

The computer program and the computer-readable storage medium according to the present embodiments may each be comprehended as a computer program product with the commands.

Further features of the present embodiments may be found in the claims, the figures, and the description of the figures.

The features and combinations of features mentioned above in the description, as well as the features and combinations of features mentioned below in the description of the figures and/or shown in the figures may be encompassed by the present embodiments not only in the respectively disclosed combination but also in other combinations. For example, embodiments and combinations of features that do not have all features of an originally worded claim may also be encompassed by the present embodiments. Further, embodiments and combinations of features that go beyond the combinations of features set out in the back references of the claims or that deviate from these may also be encompassed by the present embodiments.

The present embodiments will be explained in more detail below based on specific example embodiments and associated schematic drawings. Same or functionally same elements may be provided with the same reference characters in the figures. The description of same or functionally same elements will optionally not necessarily be repeated with respect to different figures.

### BRIEF DESCRIPTION OF THE DRAWINGS

FIG. 1 shows a schematic representation of an example embodiment of a system for visual support in a medical intervention on a hollow organ of a patient;

FIG. 2 shows a schematic flowchart of an example embodiment of a method for visual support in a medical intervention on a hollow organ of a patient;

FIG. 3 shows a schematic representation of an abdominal aorta and a vessel transplant in the abdominal aorta;

FIG. 4 schematically shows a reference representation of a pre-operative reference representation of a hollow organ and a reference volume according to a further example embodiment; and

FIG. 5 shows a visualization of an act in a tomosynthesis method according to a further example embodiment of a method.

### DETAILED DESCRIPTION

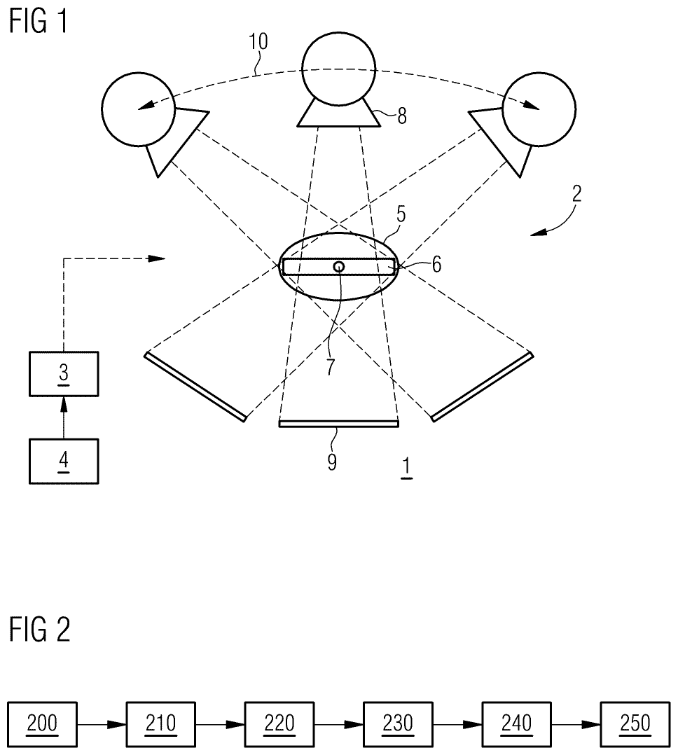

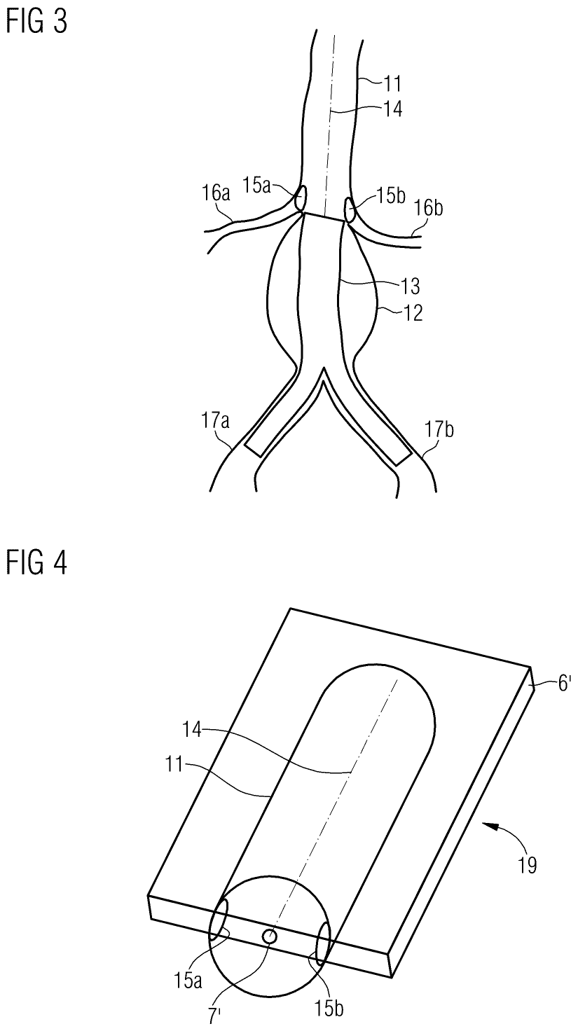

FIG. 1 schematically represents an example embodiment of a system 1 for visual support in a medical intervention on a hollow organ 11 (see FIG. 3) of a patient 5. The system 1 has an imaging modality 2.

The imaging modality 2 may be configured, for example, as a C-arm X-ray angiography system. The imaging modality 2 has, for example, an X-ray source 8, an X-ray detector, and a drive apparatus for positioning the X-ray source 8 and the X-ray detector 9 with respect to a recording region for positioning the patient 5 or the hollow organ 11 corresponding to different projection directions. The recording region may correspond, for example, to a region on a patient couch or the like, which may be recorded by the X-ray source 8 and the X-ray detector 9.

A projection direction may be specified, for example, by an angle that encloses a connecting line between the X-ray source 8 and the X-ray detector 9 with a reference direction (e.g., a horizontal in the representation in FIG. 1). The X-ray source 8 and the X-ray detector 9 may be rotated by the drive apparatus about an axis of rotation that, in the representation in FIG. 1, is, for example, perpendicular to the drawing plane, as indicated in FIG. 1 by the direction of rotation 10.

Further, the system 1 has at least one computing unit 3 that, first, may actuate the X-ray source 8 for emitting X-ray radiation and, second, may receive corresponding detector signals or detector data from the X-ray detector 9 that correspond to a projection image corresponding to the instantaneous projection direction. The at least one computing unit 3 may also actuate the drive apparatus in order to set the different projection directions. For example, the system 1 may also have a display device 9 that may likewise be actuated by the at least one computing unit 3.

A method of the present embodiments may be carried out by the system 1. FIG. 2 represents a schematic flowchart of an example embodiment of such a method.

In act 200, the at least one computing unit 3 may receive reference data that includes a reference representation 19 (see FIG. 4) of the hollow organ 11. The reference data also include items of planning information relating to a location of at least one characteristic feature 14, 15a, 15b (see FIG. 3) of the hollow organ 11. In act 210, the at least one computing unit 3 determines a reference volume 6′ (see FIG. 4) in the reference representation 19 as a function of the items of planning information.

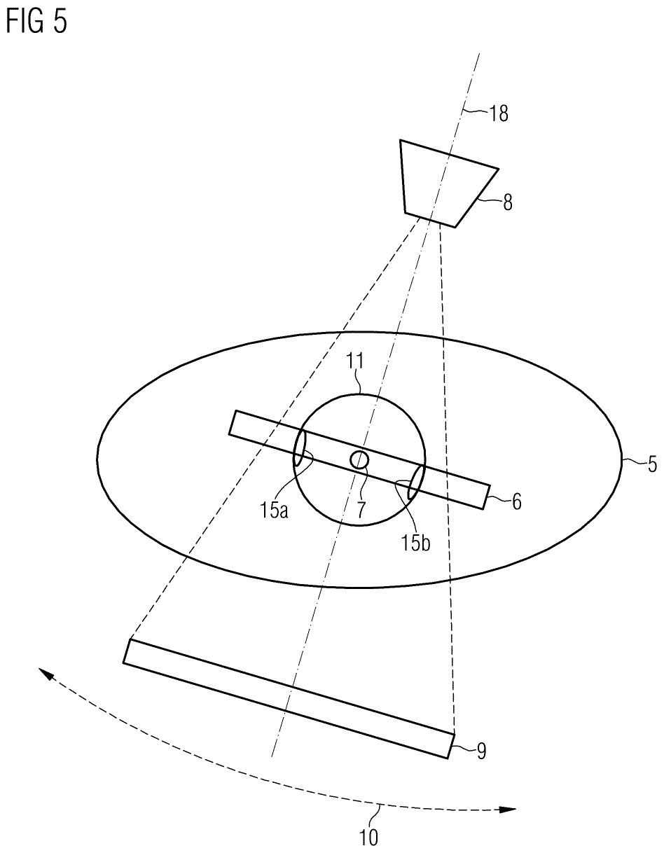

In act 220, for example, a pre-registration of the reference representation 19 may be carried out in relation to the patient 5. Starting from the pre-registration, the at least one computing unit 3 actuates the X-ray imaging modality 2 in act 230 for carrying out a tomosynthesis method, and a reconstruction 6 (see FIG. 5) of a volume of the patient 5 corresponding to the reference volume 6′ is generated based on a result of the tomosynthesis. In act 240, the at least one computing unit 3 generates registration data. For this, the at least one computing unit 3 registers the reference volume 6′ in relation to the reconstruction 6.

In act 250, the X-ray imaging modality 2, actuated by the at least one computing unit 3, generates an X-ray projection image of the hollow organ 11, and the at least one computing unit 3 determines items of overlay information based on the reference data. The at least one computing unit 3 generates an overlay image that corresponds to the X-ray projection image overlaid with the items of overlay information as a function of the registration data. The at least one computing unit 3 may actuate the display device 4, for example, so the display device 4 displays the overlay image.

As FIG. 3 schematically represents, the hollow organ 11 may be, for example, the abdominal aorta of the patient 5. The medical intervention may serve, for example, the implantation of a vessel transplant 13 in the region of an abdominal aortic aneurysm 12 between the bifurcation of the abdominal aorta in the two pelvic arteries 17a, 17b and the vascular outlets 15a, 15b of the renal arteries 16a, 16b. FIG. 4 shows a corresponding reference representation 19 (e.g., a segmentation of the abdominal aorta based on a CT reconstruction). In this case, the characteristic features may be given, for example, by the central line 14 of the abdominal aorta and the two vascular outlets 15a, 15b.

The reference volume 6′ is determined, for example, as a planar slice with a particular slice thickness. The planar slice is determined, for example, such that the planar slice incorporates or covers the vascular outlets 15a, 15b as well as the cross-section of the abdominal aorta. For example, the reference volume 6′ may be determined such that a center 7′ of the reference volume 6′ lies on the line 14 running centrally through the vessel 11.

Correspondingly, a parameter set for carrying out the tomosynthesis method may be determined based on the definition of the reference volume 6′. The parameter set includes a recording angle range, as a function of the slice thickness of the planar slice of the reference volume 6′ is determined. Further, the parameter set includes, for example, a reference recording direction 18 that is also referred to as a tomosynthesis vector and defines the center of the rotation in the tomosynthesis method, corresponding to a predefined reference angle of the recording angle range, which is determined in accordance with the orientation of the planar slice of the reference volume 6′ with respect to the reference representation 19. The parameter set may also include an isocenter position 7 that is determined as a function of the position of the planar slice of the reference volume 6′ with respect to the reference representation 19 and is set, for example, by positioning the patient 5 relative to the X-ray source 8 and the X-ray detector 9. The vascular outlets 15a, 15b may be roughly oriented in advance by the pre-registration. The tomosynthesis is then carried out in accordance with the parameter set, and the reconstruction 6 of the volume corresponding to the reference volume 6′ is generated on the basis thereof, as schematically represented in FIG. 5.

As described, for example, with reference to the figures, the present embodiments make it possible to visually support medical staff in a medical intervention on a hollow organ of a patient with reduced outlay.

In different embodiments, this is achieved, for example, by a registration of a reference volume from a pre-operative 3D reference representation in relation to a tomosynthesis volume. For example, neither administering of a contrast medium nor a complete three-dimensional (3D) run within the meaning of a CT method is required for the registration. This is achieved, for example, by taking into account the items of planning information for the definition of the reference volume, which in turn supplies the necessary parameters for tomosynthesis.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for visual support in a medical intervention on a hollow organ of a patient, the method comprising:
obtaining reference data that includes a pre-operative reference representation of the hollow organ, wherein the reference data includes items of planning information relating to a location of at least one characteristic feature of the hollow organ in the pre-operative reference representation;
generating an X-ray projection image of the hollow organ;
determining items of overlay information based on the reference data;
displaying, by a display device, an overlay image that corresponds to the X-ray projection image overlaid with the items of overlay information;
determining a reference volume in the pre-operative reference representation as a function of the items of planning information;
generating a reconstruction of a volume of the patient corresponding to the reference volume using a tomosynthesis method;
generating registration data, the generating of the registration data comprising registering the reference volume in relation to the reconstruction;
transforming the X-ray projection image or the items of overlay information in accordance with the generated registration data; and
overlaying the items of overlay information on the transformed X-ray projection image or overlaying the transformed items of overlay information on the X-ray projection image.

2. The method of claim 1, wherein the reference volume is determined as a planar slice, and
wherein the method further comprises determining a slice thickness of the planar slice and a location of the planar slice in the pre-operative reference representation for determining the reference volume.

3. The method of claim 2, further comprising determining a parameter set for carrying out the tomosynthesis method as a function of the reference volume,
wherein the tomosynthesis method is carried out in accordance with the parameter set.

4. The method of claim 3, wherein:
the parameter set includes a recording angle range, and the recording angle range is determined as a function of the slice thickness;
the parameter set includes a reference recording direction corresponding to a predefined reference angle of the recording angle range, and the reference recording direction is determined as a function of the location of the planar slice;
the parameter set includes an isocenter position, and the isocenter position is determined as a function of the location of the planar slice; or
any combination thereof.

5. The method of claim 4, wherein carrying out the tomosynthesis method comprises:
generating a sequence of further X-ray projection images, wherein each X-ray projection image of the sequence of further X-ray projection images at least partially represents the hollow organ according to another predefined projection direction, respectively, wherein the other predefined projection directions of the further X-ray projection images of the sequence are characterized by a respective angle within the recording angle range in a plane, wherein the reference recording direction lies in the plane, and
wherein generating the reconstruction comprises generating the reconstruction based on the sequence of further X-ray projection images.

6. The method of claim 4, wherein a maximum angle difference according to the recording angle range is less than 180°, is at most 90°, is at most 60°, or is at most 45°.

7. The method of claim 1, wherein the hollow organ contains a vessel, and the at least one characteristic feature includes a line running centrally through the vessel.

8. The method of claim 7, wherein the reference volume is determined so a center of the reference volume lies on the line running centrally through the vessel.

9. The method of claim 1, wherein the hollow organ contains a vessel, and the at least one characteristic feature includes a first vascular outlet on the vessel.

10. The method of claim 9, wherein the at least one characteristic feature includes a second vascular outlet on the vessel, and the reference volume is determined so the reference volume respectively at least partially includes the first vascular outlet and the second vascular outlet.

11. The method of claim 1, further comprising before carrying out the tomosynthesis method, carrying out a pre-registration of the reference representation in relation to the patient.

12. The method of claim 11, wherein carrying out the pre-registration comprises:
generating a first reference X-ray projection image of the hollow organ; and
registering the reference representation in relation to the first reference X-ray projection image.

13. The method of claim 12, wherein carrying out the pre-registration further comprises:
generating a second reference X-ray projection image of the hollow organ, wherein a projection direction for generating the first reference X-ray projection image and a projection direction for generating the second reference X-ray projection image are different from each other; and
registering the reference representation in relation to the second reference X-ray projection image.

14. A system for visual support in a medical intervention on a hollow organ of a patient, the system comprising:
an X-ray imaging modality that is configured to generate an X-ray projection image of the hollow organ; and
at least one computing unit that is configured to:
obtain reference data that includes a reference representation of the hollow organ, wherein the reference data includes items of planning information relating to a location of at least one characteristic feature of the hollow organ in the reference representation;
determine items of overlay information based on the reference data;
generate an overlay image that corresponds to the X-ray projection image overlaid with the items of overlay information;
determine a reference volume in the reference representation as a function of the items of planning information;
actuate the X-ray imaging modality for carrying out a tomosynthesis method;
generate a reconstruction of a volume of the patient corresponding to the reference volume based on a result of the tomosynthesis method;
generate registration data;
register the reference volume in relation to the reconstruction;
transform the X-ray projection image or the items of overlay information in accordance with the generated registration data; and
overlay the items of overlay information on the transformed X-ray projection image or overlay the transformed items of overlay information on the X-ray projection image.

15. (canceled)

16. The method of claim 1, wherein generating registration data comprises generating 3D/3D registration data.

17. The method of claim 1, wherein the items of overlay information comprise a two-dimensional representation of the hollow organ or another organ of the patient obtained from the pre-operative reference representation, two-dimensional representations of the characteristic features, or the two-dimensional representation of the hollow organ or the other organ of the patient obtained from the pre-operative reference representation and the two-dimensional representations of the characteristic features.
